(19) United States
(12) Patent Application Publication
(10) Pub. No.: US 2024/0024211 A1
(43) Pub. Date: Jan. 25, 2024

(54) WATER-IN-OIL EMULSION COMPOSITION INCLUDING SALICYLIC ACID

(71) Applicant: Shiseido Company, Ltd., Chuo-ku, Tokyo (JP)

(72) Inventor: Megumi YOSHIDA, Tokyo (JP)

(73) Assignee: Shiseido Company, Ltd., Chuo-ku, Tokyo (JP)

(21) Appl. No.: 18/028,675

(22) PCT Filed: Oct. 30, 2020
(86) PCT No.: PCT/JP2020/040742

§ 371 Date: Mar. 27, 2023

(51) Int. Cl. A61K 8/06; A61K 8/365; A61Q 1/00

(52) U.S. Cl. CPC A61K 8/064; A61K 8/365; A61Q 1/00; A61K 2800/43

(57) ABSTRACT

The purpose of the present invention is to provide a water-in-oil emulsion composition that is stable over a long period of time without allowing syneresis to occur, even when a relatively large quantity of salicylic acid has been blended therein, and furthermore does not lose usability over time and can be applied in a make-up cosmetic. The present invention relates to a water-in-oil emulsion composition that is characterized by including: (a) 0.5 mass % or more of salicylic acid; (b) an organic modified clay mineral; (c) a sorbitan fatty acid ester; and (d) a coloring material.

### TECHNICAL FIELD

The present invention relates to a water-in-oil emulsion composition. In particular, the present invention relates to a water-in-oil emulsion composition that can stably contain a relatively large amount of salicylic acid, and an emulsion cosmetic using the same.

### BACKGROUND ART

Salicylic acid is known as an effective compound for healing relatively mild acne. Further, when applied to the skin, salicylic acid derivatives, such as 4-methoxysalicylate, exhibit a whitening effect that improves or prevents abnormal deposition of melanin pigment. However, salicylic acid or derivatives thereof in water-in-oil emulsion compositions may cause a problem that coalescence of emulsified particles occurred over time to make the system unstable, resulting in separation (syneresis).

Patent Document 1 discloses a water-in-oil-type skin-whitening cosmetic comprising 4-methoxysalicylate, which is a salicylic acid derivative. This cosmetic is blended with a combination of an organically modified clay mineral and oleic acid and/or isostearic acid, and thus has a moist feel without stickiness when used and good stability over time regardless of the container material.

However, when 4-methoxysalicylate in the cosmetic of Patent Document 1 was replaced by salicylic acid, the viscosity of the system tended to increase over time and texture tended to be heavy. Further, the cosmetic of Patent Document 1 is a skin care cosmetic for whitening, and is not intended to be blended with coloring materials (powder), such as pigments. In fact, Patent Document 1 does not refer to coloring materials, and no coloring material is blended in the specifically described formulas. Therefore, it is not clear what change will occur when trying to blend a coloring material in the cosmetic of Patent Document 1 and applying it to a makeup cosmetic.
Patent Document 1: JP-B 5164292

### SUMMARY OF INVENTION

### Technical Problem

Therefore, an object of the present invention is to provide a water-in-oil emulsion composition that stable and does not undergo syneresis for a long period of time even if a relatively large amount of salicylic acid is blended, that does not deteriorate feeling of use over time, and that are suited for makeup cosmetics.

### Solution to Problem

As a result of extensive studies to achieve the above object, the present inventor found that a water-in-oil emulsion composition that is stable for a long period of time and that can maintain excellent texture can be obtained by blending a sorbitan fatty acid ester in a water-in-oil emulsion composition comprising at least 0.5% by mass of salicylic acid, an organically modified clay mineral, and a coloring material. Thus, the present invention has been completed.

Specifically, the present invention provides a water-in-oil emulsion composition comprising:
(a) 0.5% by mass or more of salicylic acid;
(b) an organically modified clay mineral;
(c) a sorbitan fatty acid ester; and
(d) a coloring material.

### Advantageous Effect of Invention

The composition of the present invention is extremely stable over time even though it contains a relatively high concentration of salicylic acid, which is considered to tend to destroy the water-in-oil emulsion state. At the same time, good texture is maintained for a long period of time with little change in viscosity over time. Furthermore, the composition does not cause color stripes and is particularly suitable as a makeup cosmetic.

### DESCRIPTION OF EMBODIMENTS

As described above, the water-in-oil emulsion composition of the present invention comprises (a) salicylic acid, (b) an organically modified clay mineral, (c) a sorbitan fatty acid ester, and (d) a coloring material as essential components. The details are provided below.

(a) Salicylic Acid

Salicylic acid is known to be effective in healing relatively mild acne, and is a well-known component that has been conventionally used in cosmetics.

Conventional cosmetics contain only a small amount of salicylic acid; however, the composition of the present invention is characterized in that at least 0.5% by mass of salicylic acid can be stably blended. The amount of salicylic acid in the composition of the present invention is 0.5% by mass or more. The upper limit of the blending amount is not particularly limited, but is generally 20% by mass or less, and preferably about 10% by mass or less.

Since salicylic acid has low solubility in water, when the composition of the present invention is prepared, it is preferable to dissolve and blend salicylic acid in a suitable amount of alcohol (ethanol).

When the salicylic acid in the composition of the present invention is replaced by a salicylic acid derivative (alkoxysalicylic acid), such as 4-methoxysalicylate, viscosity tends to increase over time, and texture tends to be heavy, which is not preferable. Therefore, “salicylic acid” in the present invention includes salicylic acid and salts thereof, but does not include salicylic acid derivatives, such as alkoxysalicylic acid.

(b) Organically Modified Clay Mineral

The organically modified clay mineral used in the composition of the present invention is preferably one obtained by adding a quaternary ammonium salt compound to a natural or synthetic smectite clay mineral, such as bentonite, by an ion-exchange reaction. The organically modified clay mineral is not particularly limited as long as it is pharmaceutically acceptable. Examples include dimethylammonium hectorite, benzyldimethylstearylammonium hectorite, magnesium aluminum silicate treated with distearyldimethylammonium chloride, and the like. Preferred examples of commercial products include dimethyldistearylammonium hectorite sold by Elementis Specialties Inc. under the name of “Bentone 38VCG,” benzyldimethylstearylammonium hectorite sold by Elementis Specialties Inc. under the name of “Bentone 27VCG,” and the like.

The organically modified clay minerals can be blended singly or in combination of two or more. The amount of organically modified clay mineral blended is preferably 0.1 to 5.0% by mass, and preferably, for example, 1.5 to 5.0% by mass, based on the total amount of the composition. If the blending amount is less than 0.1% by mass, emulsion stability tends to decrease, whereas if the blending amount exceeds 5.0% by mass, stickiness occurs, and texture tends to be inferior.

(c) Sorbitan Fatty Acid Ester

The sorbitan fatty acid ester used in the composition of the present invention is an ester of C₁₂₋₂₀ fatty acid and sorbitol. Examples of the C₁₂₋₂₀ fatty acid include stearic acid, isostearic acid, sesquiisostearic acid, and the like. Specific examples include sorbitan monoisostearate, sorbitan sesquiisostearate, and the like. Of these, sorbitan sesquiisostearate is preferably used. Examples of commercial products include “Estemol 182V” (produced by The Nisshin OilliO Group, Ltd.), “Emalex SPIS-150” (produced by Nihon Emulsion Co., Ltd.), and the like.

The amount of sorbitan fatty acid ester in the composition of the present invention is generally 0.1 to 4.0% by mass, preferably 0.5 to 3.0% by mass, and more preferably 0.5 to 1.5% by mass. If the blending amount is less than 0.1% by mass, sufficient emulsion stability cannot be obtained, whereas if the blending amount exceeds 4.0% by mass, stickiness may occur.

Patent Document 1 relates to an invention that improves emulsion stability by blending oleic acid and/or isostearic acid in a system in which potassium 4-methoxysalicylate, which is a salicylic acid derivative, and an organically modified clay mineral coexist. Patent Document 1 indicates that in addition to liquid fatty acids, such as isostearic acid, an emulsifier with a low HLB (7 or less) can be blended (paragraph 0024), and examples of the low-HLB emulsifier include sorbitan fatty acid esters. However, it was first found that when the salicylic acid derivative in the system of Patent Document 1 was replaced by salicylic acid and a coloring material was further blended, viscosity changes over time were observed even when isostearic acid was blended, that by blending a sorbitan fatty acid ester in a system in which salicylic acid, an organically modified clay mineral, and a coloring material coexist, an extremely stable composition can be obtained without viscosity changes over time even if it does not contain liquid fatty acids. Therefore, the composition of the present invention includes an embodiment in which it does not contain liquid fatty acids, such as isostearic acid and oleic acid.

(d) Coloring Material

As the coloring material in the composition of the present invention, pigments, pearl pigments, and the like that are conventionally used in makeup cosmetics can be used.

Specifically, one or two or more members can be selected from, but are not limited to, inorganic white pigments (titanium dioxide and zinc oxide), inorganic red pigments (iron oxide (red iron oxide) and iron titanate), inorganic brown pigments (γ-iron oxide), inorganic yellow pigments (yellow iron oxide and ocher), inorganic black pigments (black iron oxide, carbon, and low-order titanium oxide), inorganic purple pigments (mango violet and cobalt violet), inorganic green pigments (chromium oxide, chromium hydroxide, and cobalt titanate), inorganic blue pigments (ultramarine and navy blue), pearl pigments (titanium oxide-coated mica, titanium oxide-coated bismuth oxychloride, titanium oxide-coated talc, colored titanium oxide-coated mica, bismuth oxychloride, and fish scale foil), metal powder pigments (aluminum powder and copper powder), organic pigments (Red 202, Red 205, Red 220, Red 228, Red 405, Orange 203, Orange 204, Yellow 205, Yellow 401, and Blue 404), zirconium, barium, or aluminum lake organic pigments (Red 3, Red 104, Red 227, Red 401, Orange 205, Yellow 4, Yellow 202, Green 3, and Blue 1), natural pigments (chlorophyll, carcinoids (8-carotene), carthamin, cochineal, chalcone, curcumin, betanin, flavonol, flavone, anthocyanidin, anthraquinone, and naphthoquinone), and functional pigments (boron nitride, photochromic pigment, synthetic fluorphlogopite, iron-containing synthetic fluorphlogopite, and fine particle composite powder (hybrid fine powder)).

As powder-like coloring materials, such as pigments, those with an untreated surface may be used. Alternatively, those with a surface treated with organosilane compounds, silicone compounds, fluorine compounds, silane coupling agents, fluororesins, fatty acids, fatty acid soaps, lauroyl lysine, or the like may also be used.

When the composition of the present invention contains 0.1% by mass or more of coloring material based on the total amount of the composition, the effect of the present invention, i.e., prevention of color stripes, can be exhibited. Therefore, the amount of coloring material in the composition of the present invention can be set to 0.1% by mass or more. In particular, when the composition of the present invention is formed into a makeup cosmetic, the amount of coloring material blended is preferably 1.0% by mass or more, more preferably 5.0% by mass or more, and even more preferably 10% by mass or more. If the blending amount is less than 1.0% by mass, a sufficient makeup effect cannot be obtained. The upper limit of the amount of coloring material blended is not particularly limited, but is generally 40% by mass or less, and preferably 30% by mass or less. If the blending amount exceeds 40% by mass, stability may be reduced.

The composition of the present invention is a water-in-oil emulsion composition. Therefore, the composition contains an oil that constitutes the outer phase of the emulsion, and water that constitutes the inner phase.

The oil that constitutes the outer phase is not particularly limited as long as it can be used for cosmetics etc.; however, silicone oil is preferably contained. As the silicone oil, volatile silicone oils, especially volatile linear silicone oils (1.5 cs, 2 cs, etc.), are particularly preferably used. The amount of oil in the composition of the present invention is about 1 to 30% by mass.

The water that constitutes the inner phase can hold hydrophilic components, such as alcohol (ethanol). The amount of water in the composition of the present invention is about 20 to 40% by mass.

The composition of the present invention may be blended with, in addition to the essential components (a) to (d) as well as oil and water, other optional components that are generally used in cosmetics, pharmaceuticals, and the like within a range that does not impair the effects of the present invention.

For example, syneresis stability can be further enhanced by blending a polyether-modified silicone in the composition of the present invention.

The polyether-modified silicone preferably used in the present invention is a polyether-modified silicone having a polyoxyalkylene group grafted on the polysiloxane skeleton, and may optionally be alkyl co-modified. Usable polyether-modified silicones are a polyether-modified silicone having a linear polysiloxane skeleton (linear) and a polyether-modified silicone having a branched polysiloxane skeleton (branched); however, in terms of improving stability, a branched polyether-modified silicone is preferred. Although it is not particularly limited, a polyether-modified silicone having an HLB of preferably 6.0 or less, more preferably 5.0 or less, and preferably 3.0 or more, and a polyether-modified silicone that is not alkyl co-modified are preferably used.

Specific examples of polyether-modified silicones include PEG-3 dimethicone, PEG-10 dimethicone, and cetyl PEG/PPG-10/1 dimethicone (all of which are linear), PEG-9 polydimethylsiloxyethyl dimethicone (which is branched), and the like. Particularly preferred among these is PEG-9 polydimethylsiloxyethyl dimethicone.

When a polyether-modified silicone is blended, the blending amount thereof is preferably 0.1 to 5% by mass, more preferably 1 to 4% by mass, and particularly preferably 2 to 3% by mass, based on the total amount of the composition. If the blending amount is less than 0.1% by mass, a stabilization enhancement effect cannot be obtained, whereas if the blending amount exceeds 5% by mass, color stripes may occur.

When polyethylene glycol is blended in the composition of the present invention, the effect of stably holding salicylic acid can be further enhanced.

The polyethylene glycol preferably used in the present invention has an average molecular weight of 1000 to 15000. Specific examples include polyethylene glycol 1000 (INCI name: PEG-20), polyethylene glycol 1500 (INCI name: PEG-6; PEG-32), polyethylene glycol 4000 (INCI name: PEG-75), polyethylene glycol 6000 (INCI name: PEG-150), polyethylene glycol 11000 (INCI name: PEG-240), and the like. Preferred among these is polyethylene glycol-6000.

The amount of polyethylene glycol blended is preferably 1 to 8% by mass, more preferably 2 to 7% by mass, and particularly preferably 3 to 6% by mass, based on the total amount of the composition.

Examples of other optional components include humectants, UV absorbers, UV scattering agents, buffers, chelating agents, preservatives, fragrances, various chemicals, and the like, but are not limited to these exemplified components.

The water-in-oil emulsion composition of the present invention can be prepared by a standard method, and the method of emulsification is not particularly limited. For example, an aqueous phase (inner phase) and an oil phase (outer phase) that are separately prepared are each heated to about 80° C., the heated aqueous phase is gradually added to the oil phase, and the mixture is emulsified with an emulsifying machine, followed by cooling to room temperature.

Since the composition of the present invention stably contains salicylic acid at a higher concentration than before, it sufficiently exhibits an anti-acne effect based on salicylic acid. In addition, coloring materials, such as pigments, are blended, and color stripes do not occur; thus, the composition of the present invention exhibits an excellent makeup effect. Therefore, the composition of the present invention is particularly suitable for provision as makeup cosmetics.

### Examples

The present invention will be described in more detail below with reference to Examples; however, the technical scope of the present invention is not limited to these Examples.

Makeup cosmetics of the formulations shown in Table 1 below were prepared according to a standard method.

The cosmetics of the examples were evaluated for syneresis stability, color stripes, and texture (immediately after preparation and after 2 weeks) according to the following methods and criteria.

(1) Syneresis Stability

After the cosmetic of each example was allowed to stand at 50° C. for 4 weeks, the occurrence of syneresis (oil float) was visually observed and evaluated according to the following criteria.

### (Evaluation Criteria)

A: No change (no syneresis) occurred.
B: A small amount of transparent liquid was observed on the surface.
C: A clear liquid pool was observed.

(2) Color stripes

The cosmetic of each example was stored in a polyethylene container, and the body of the cosmetic was visually observed and evaluated according to the following criteria.

### (Evaluation Criteria)

A: No color stripes
B: Blurred color stripes in a narrow area, but no problem with the overall appearance
C: Blurred color stripes in a wide area

(3) Texture

The texture of the cosmetic of each example was evaluated by expert panels immediately after preparation (immediate) and after standing at room temperature for 2 weeks (over time) according to the following criteria.

### (Evaluation Criteria)

A: Easy to spread
B: Slightly stretchy
C: Very stretchy

TABLE 1

| | | Comparative | Comparative | Comparative | Comparative |
|---|---|---|---|---|---|
| | Example 1 | Example 1 | Example 2 | Example 3 | Example 4 |
| Dimethicone (2 cs) | 16 | 16 | 16 | 16 | 16 |
| Dimethicone (6 cs) | 1 | 1 | 1 | 1 | 1 |
| Cetyl ethylhexanoate | 5 | 5 | 5 | 5 | 5 |
| PEG-9 | 3 | 3 | 3 | 3 | 3 |
| polydimethylsiloxyethyl | | | | | |
| dimethicone | | | | | |
| Sorbitan | 1 | — | — | — | 1 |
| sesquiisostearate | | | | | |
| Isostearic acid | — | 1 | — | — | — |
| Disteardimonium | 2 | 2 | 2 | 2 | 2 |
| hectorite | | | | | |
| Silicone-treated pigment- | 18 | 18 | 18 | 18 | 18 |
| grade titanium dioxide | | | | | |
| Silicone-treated red iron | 1.3 | 1.3 | 1.3 | 1.3 | 1.3 |
| oxide | | | | | |
| Silicone-treated yellow | 2 | 2 | 2 | 2 | 2 |
| iron oxide | | | | | |
| Silicone-treated black | 0.15 | 0.15 | 0.15 | 0.15 | 0.15 |
| iron oxide | | | | | |
| Purified water | 37.67 | 37.67 | 38.67 | 39.37 | 37.87 |
| Potassium hydroxide | 0.2 | 0.2 | 0.2 | — | — |
| EDTA-3Na•2H2O | 0.2 | 0.2 | 0.2 | 0.2 | 0.2 |
| Citric acid | 0.02 | 0.02 | 0.02 | 0.02 | 0.02 |
| Sodium citrate | 0.06 | 0.06 | 0.06 | 0.06 | 0.06 |
| Ethanol | 5 | 5 | 5 | 5 | 5 |
| Salicylic acid | 0.5 | 0.5 | 0.5 | — | — |
| Potassium 4- | — | — | — | — | 0.5 |
| methoxysalicylate | | | | | |
| Preservative | 0.5 | 0.5 | 0.5 | 0.5 | 0.5 |
| PEG-150 | 5 | 5 | 5 | 5 | 5 |
| Glycerol | 1.4 | 1.4 | 1.4 | 1.4 | 1.4 |
| Syneresis stability | A | A | A | A | A |
| Color stripes | A | A | B | B | C |
| Texture (immediate) | A | A | C | B | A |
| Texture (over time) | A | B | C | B | B |

As shown in Table 1, the cosmetic of Example 1, which was a water-in-oil emulsion composition containing all of (a) salicylic acid, (b) an organically modified clay mineral, (c) a sorbitan fatty acid ester, and (d) a coloring material, had excellent syneresis stability, did not form color stripes, and did not undergo changes in texture overtime. On the other hand, in Comparative Example 1, in which the sorbitan fatty acid ester was replaced by isostearic acid, the viscosity increased over time, and texture became slightly heavy. In Comparative Example 2, in which neither a sorbitan fatty acid ester nor isostearic acid was blended, texture was heavy immediately after preparation, and some color stripes occurred. In Comparative Example 3, in which salicylic acid was removed from Comparative Example 2, texture was slightly improved, but color stripes were not suppressed. Further, in Comparative Example 4, in which the salicylic acid in Example 1 was replaced by 4-methoxysalicylic acid, the viscosity increased over time, texture became slightly heavy, and significant color stripes occurred. These results supported that blending all of the essential components (a) to (d) of the present invention can not only ensure syneresis stability, but also suppress color stripes and texture changes (viscosity increase).

1. A water-in-oil emulsion composition comprising:
(a) 0.5% by mass or more of salicylic acid;
(b) an organically modified clay mineral;
(c) a sorbitan fatty acid ester; and
(d) a coloring material.

2. The composition according to claim 1, wherein the amount of the organically modified clay mineral (b) is 0.1 to 5.0% by mass based on the total amount of the composition.

3. The composition according to claim 1, further comprising a polyether-modified silicone.

4. The composition according to claim 1, further comprising polyethylene glycol.

5. The composition according to claim 1, wherein the sorbitan fatty acid ester (c) is sorbitan sesquiisostearate.

6. A makeup cosmetic comprising the composition according to claim 1.
